(19) United States
(12) Patent Application Publication
(10) Pub. No.: US 2024/0020828 A1
(43) Pub. Date: Jan. 18, 2024

(54) MACHINE LEARNING SYSTEM FOR X-RAY BASED IMAGING CAPSULE

(71) Applicant: CHECK-CAP LTD., isfiya (IL)

(72) Inventor: Yoav KIMCHY, Haifa (IL)

(21) Appl. No.: 18/034,713

(22) PCT Filed: Dec. 30, 2021
(86) PCT No.: PCT/IL2021/051554

§ 371 Date: May 1, 2023

Related U.S. Application Data
(60) Provisional application No. 63/132,499, filed on Dec. 31, 2020.

(51) Int. Cl. G06T 7/00; A61B 6/00; G16H 10/60; G16H 50/20; G06V 10/764; G06V 10/774

(52) U.S. Cl. CPC G06T 7/0012; A61B 6/4057; A61B 6/485; G16H 10/60; G16H 50/20; G06V 10/764; G06V 10/774; G06T 2207/20076; G06T 2207/10116; G06T 2207/30092; G06T 2207/20084; G06T 2207/20081; G06V 2201/03

(57) ABSTRACT

A system for gastrointestinal examination, including an imaging capsule configured to scan the gastrointestinal tract of a patient using radiation and configured to measure X-ray fluorescence and Compton backscattering and transmit the measurements, a computer with a processor and memory configured to receive the measurements from the imaging capsule, a trained machine learning module executed by the computer configured to analyze the measurements and provide a probability score representing the probability of the existence of a polyp or other abnormalities in the gastrointestinal tract of the patient.

### RELATED APPLICATIONS

This application claims priority under 35 U.S.C. 120 from U.S. provisional application No. 63/132,499 dated Dec. 31, 2020, the disclosure of which is incorporated herein by reference.

### FIELD OF THE DISCLOSURE

The present disclosure relates generally to identifying polyps with an x-ray based imaging capsule and more specifically to using a machine learning system to identify the polyps from data recorded by the x-ray-based imaging capsule.

### BACKGROUND OF THE DISCLOSURE

One method for examining the gastrointestinal tract for the existence of polyps and other clinically relevant features that may indicate regarding the potential of cancer is performed by swallowing an imaging capsule that travels through the gastrointestinal tract and view the patient's situation. In a typical case the trip can take between 24-48 hours after which the imaging capsule exits in the patient's feces. Typically, the patient swallows a contrast agent to enhance the imaging ability of the imaging capsule. Then, the patient swallows the imaging capsule to examine the gastrointestinal tract while flowing through the contrast agent. In an exemplary case the imaging capsule is designed to measure X-Ray fluorescence and/or Compton back-scattering and transmits the measurements (e.g. count rate) to an external analysis device, for example a computer or other dedicated instruments. U.S. Pat. No. 7,787,926 to Kinichy the disclosure of which is incorporated herein by reference, describes details related to the manufacture and use of such an imaging capsule.

After traversing the patients gastrointestinal tract the collected measurements can then be used to form simulated 2D and/or 3D images of the patient's colon or other organs. The patient's colon is not a perfect cylinder but rather continuously varies in size and shape, therefore a trained practitioner is generally required to examine the images to identify if the patient has a polyp or other clinical relevant features.

It should be noted that a practitioner may also identify other gastrointestinal malignancies such as IBD, IBS, Crohn's Disease and other gastrointestinal inflammation using the images.

### SUMMARY OF THE DISCLOSURE

An embodiment of the disclosure, relates to a system and method of gastrointestinal examination, where measurement of a radiation based imaging capsule to identify polyps or abnormalities in a patient's gastrointestinal tract, especially the patient' colon. Data from cases that were tested with an imaging capsule and a colonoscopy test are used as input to a neural network to train machine learning module. The trained machine learning module is enabled to analyze measurements from an imaging capsule and determine if the patient has polyps or is clean.

There is thus provided according to an embodiment of the disclosure, a system for gastrointestinal examination, comprising:

An imaging capsule configured to scan the gastrointestinal tract of a patient using radiation and configured to measure X-ray fluorescence and Compton backscattering and transmit the measurements;

A computer with a processor and memory configured to receive the measurements from the imaging capsule;

A trained machine learning module executed by the computer configured to analyze the measurements and provide a probability score representing the probability of the existence of a polyp or other abnormalities in the gastrointestinal tract of the patient.

In an embodiment of the disclosure, the machine learning module considers also measurements of the internal pressure within the imaging capsule to calculate the probability score. Optionally, the machine learning module considers also capsule dynamics to calculate the probability score; wherein the capsule dynamics are selected from the group consisting of: average speed within the colon, average speed within a specific segment, capsule angular direction as a function of time, capsule location as a function of time, capsule position velocity and capsule angular velocity. In an embodiment of the disclosure, the machine learning module considers also colonoscopy information of the patient to calculate the probability score; wherein the colonoscopy information is selected from the group consisting of: if the patient had polyps in the past, if the patient has colon cancer, the whole gut transfer time of the imaging capsule. Optionally, the machine learning module considers also demographic information of the patient to calculate the probability score; wherein the demographic information is selected from the group consisting of: patient age, patient gender, height, weight, medical history, if the patient smokes, if the patient has first degree relatives that, had cancer, number of bowel movements per week, if the patient eats vegetables.

In an embodiment of the disclosure, the machine learning module is trained by a neural network that is provided with a data set comprising multiple instances of X-ray fluorescence measurements and Compton backscattering measurements for a slice of the patients colon, with a determination if the measurements indicate that the slice comprises a polyp. Alternatively, the machine learning module is trained by a neural network that is provided with a data set comprising multiple instances of X-ray fluorescence measurements and Compton backscattering measurements for a sequence of slices forming a segment of the patients colon, with a determination if the measurements indicate that the segment comprises a polyp. Optionally, the neural network considers correlation between the slices in the sequence. In an embodiment of the disclosure, the machine learning module is configured to discriminate between polyps and air bubbles based on the X-ray fluorescence measurements and Compton backscattering measurements. Optionally, the machine learning module is trained by classifying a first data set with an expert classifier, training a first machine learning module, classifying a new data set with the first machine learning module, checking at least some of the classified new data set with the expert classifier and training an improved machine learning module.

There is further provided according to an embodiment of the disclosure, a method of examining a gastrointestinal tract, comprising:

scanning the gastrointestinal tract of a patient with an imaging capsule using radiation by measuring X-ray fluorescence and Compton backscattering and transmitting the measurements;

receiving the measurements from the imaging capsule by a computer with a processor and memory;

analyzing the measurements with a trained machine learning module executed by the computer and providing a probability score representing the probability of the existence of a polyp or other abnormalities in the gastrointestinal tract of the patient.

BRIEF DESCRIPTION OF THE DRAWINGS

The present disclosure will be understood and better appreciated from the following detailed description taken in conjunction With the drawings. Identical structures, elements or parts, which appear in more than one figure, are generally labeled with the same or similar number in all the figures in which they appear, wherein:

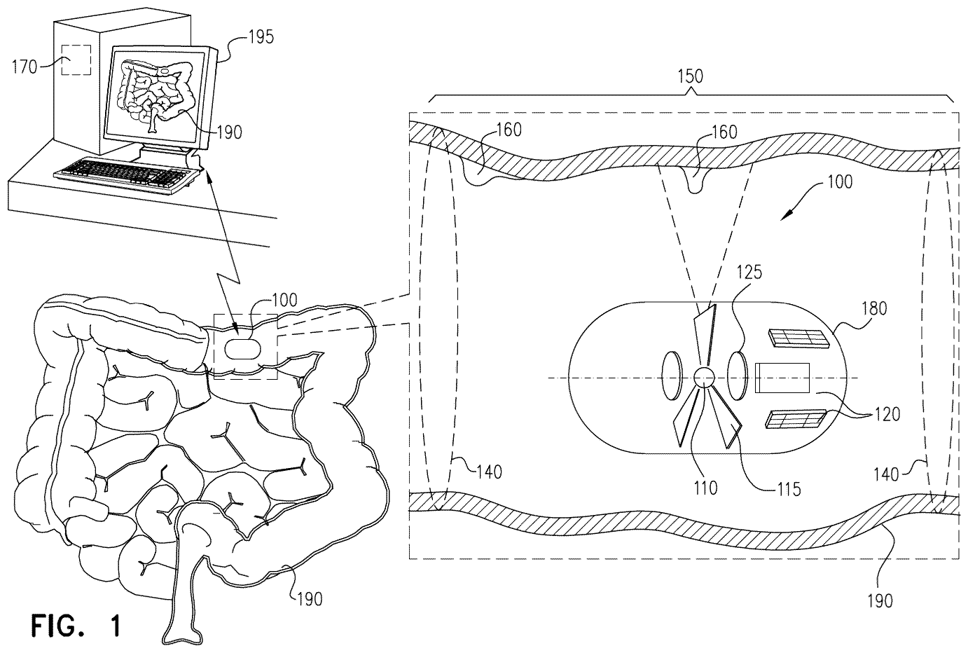

FIG. 1 is a schematic illustration of a perspective view of an imaging capsule 100 in a patient's colon 190, according to an embodiment of the disclosure;

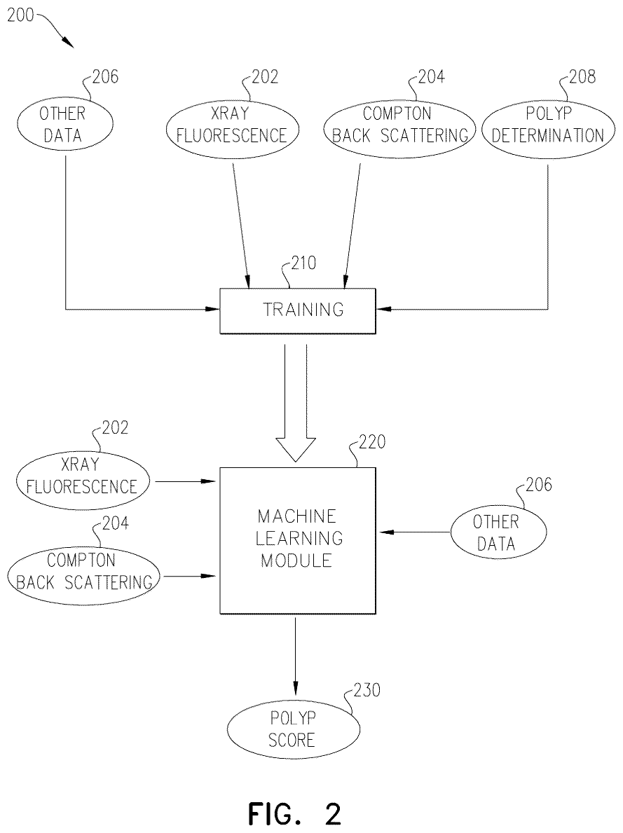

FIG. 2 is a schematic flow diagram of a process 200 of training 210 a machine learning module 220, according to an embodiment of the disclosure;

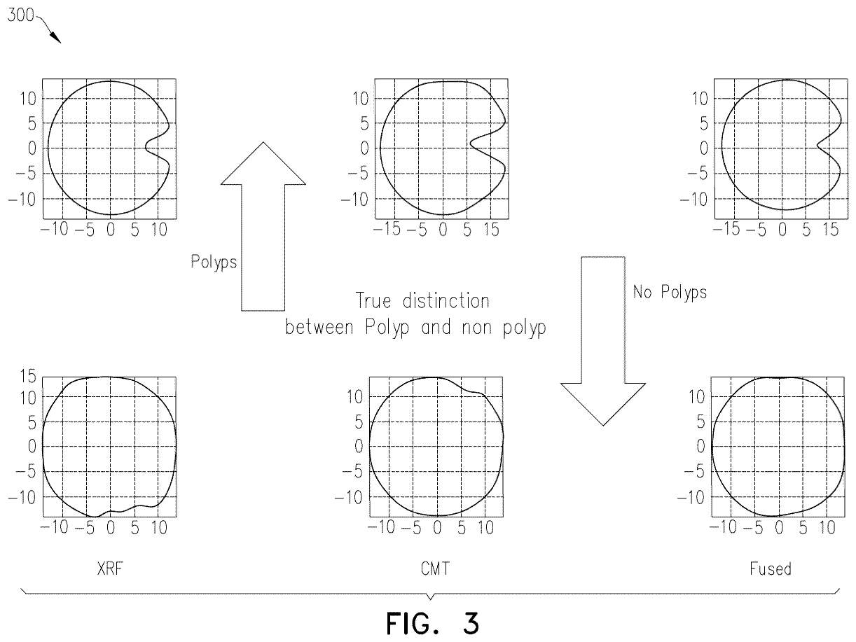

FIG. 3 is a schematic illustration of a lab generated set 300 of polyp and non-polyp 2D maps produced from the capsule imaging data; according to an embodiment of the disclosure;

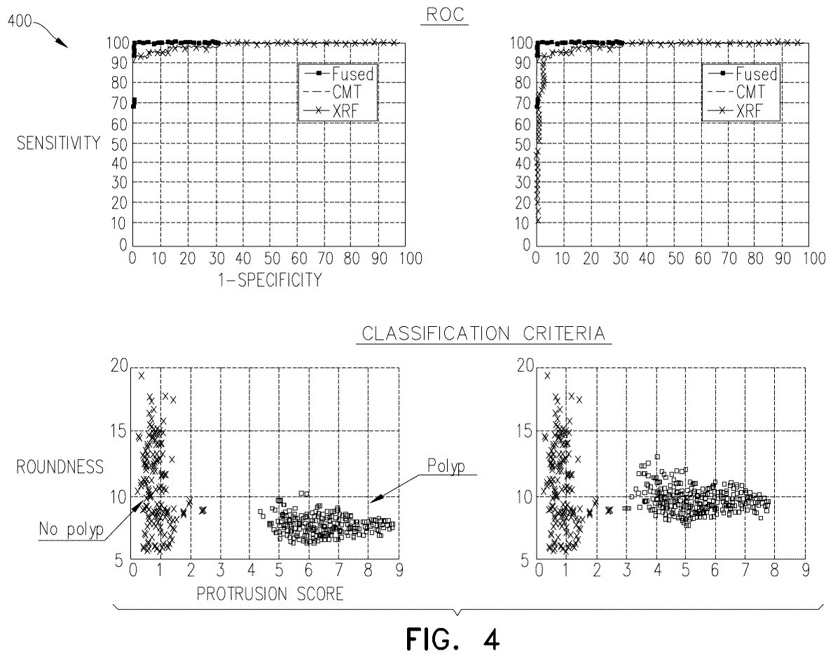

FIG. 4 is a schematic illustration of an exemplary set of a Receiver Operator Curve (ROC) and performance of a human expert classifier, according to an embodiment of the disclosure;

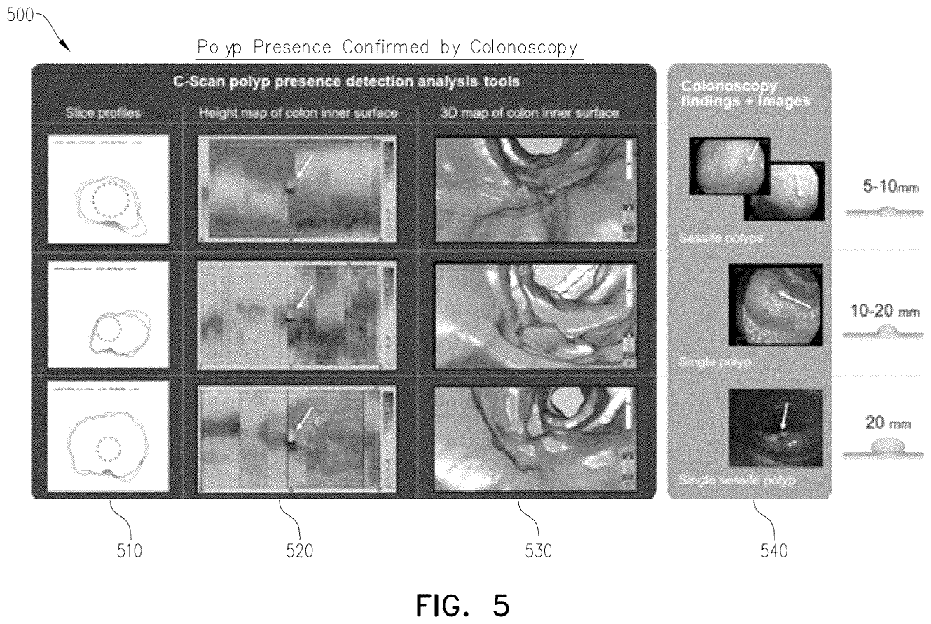

FIG. 5 is a schematic illustration of 2D and 3D images of polyps, the image of the polyp in a colonoscopy procedure and the 2D sector slice of the polyp, according to an embodiment of the disclosure;

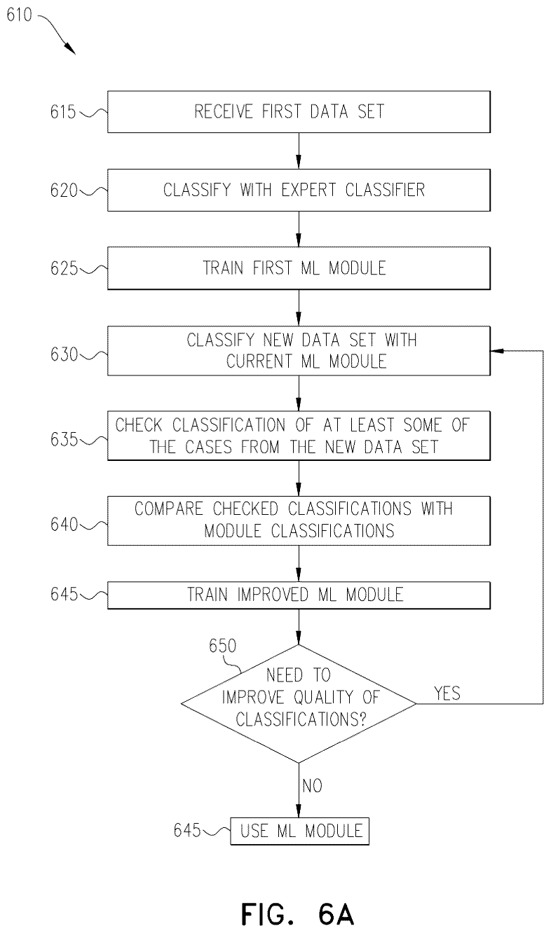

FIG. 6A is a flow diagram of a method of training a machine learning module, according to an embodiment of the disclosure;

FIG. 6B is a. table of data, some which are labelled and some not labelled, forming a semi-supervised model, according to an embodiment of the disclosure:

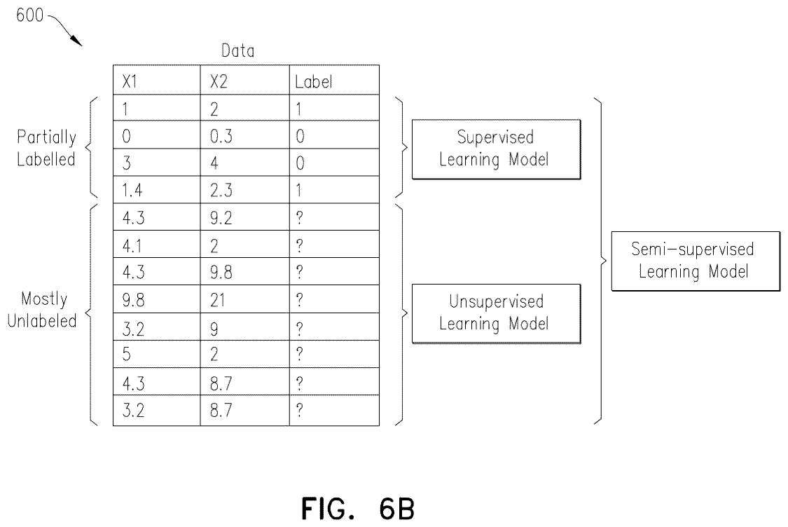

FIG. 7 is a schematic illustration of an artificial neural network for generating a machine learning module, according to an embodiment of the disclosure;

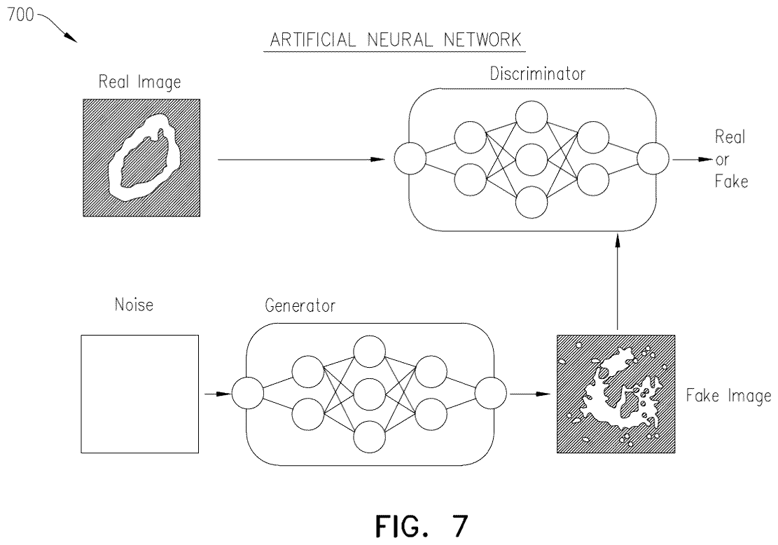

FIG. 8 is a schematic illustration of a generative adversial network (GAN), according to an embodiment of the disclosure;

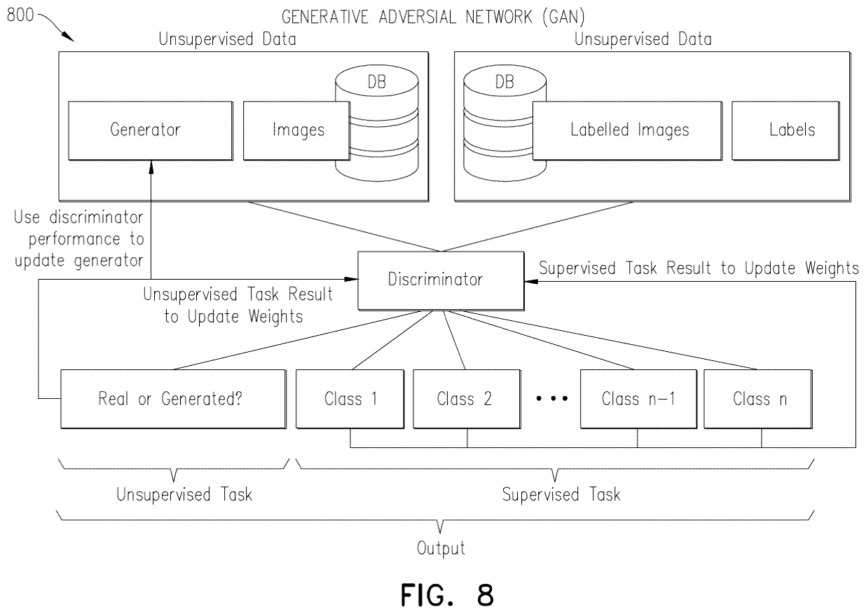

FIG. 9 is a schematic illustration of a conditional GAN classifier, according to an embodiment of the disclosure;

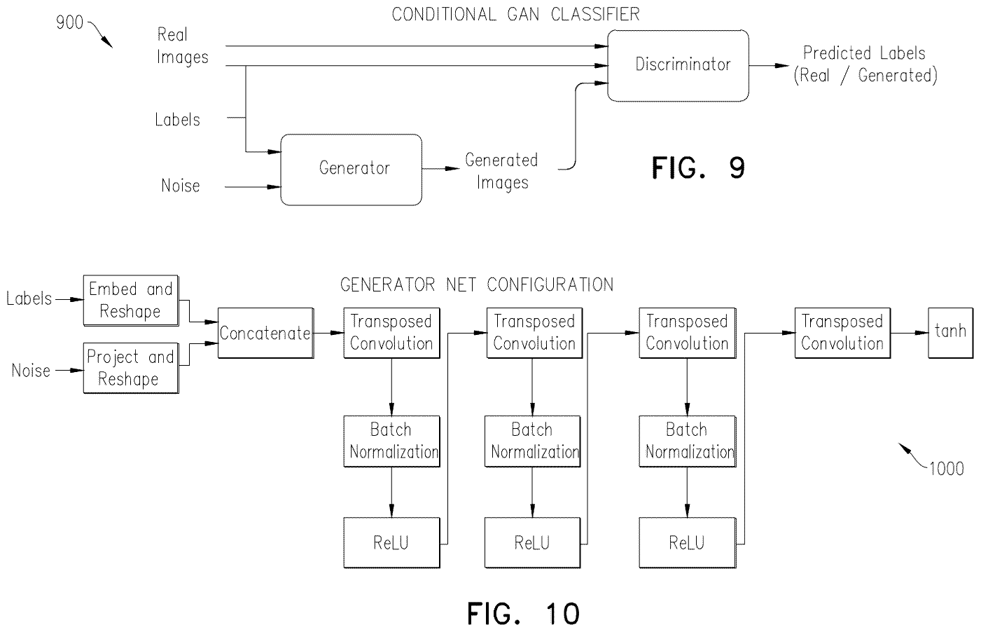

FIG. 10 is a schematic illustration of a generator net configuration, according to an embodiment of the disclosure;

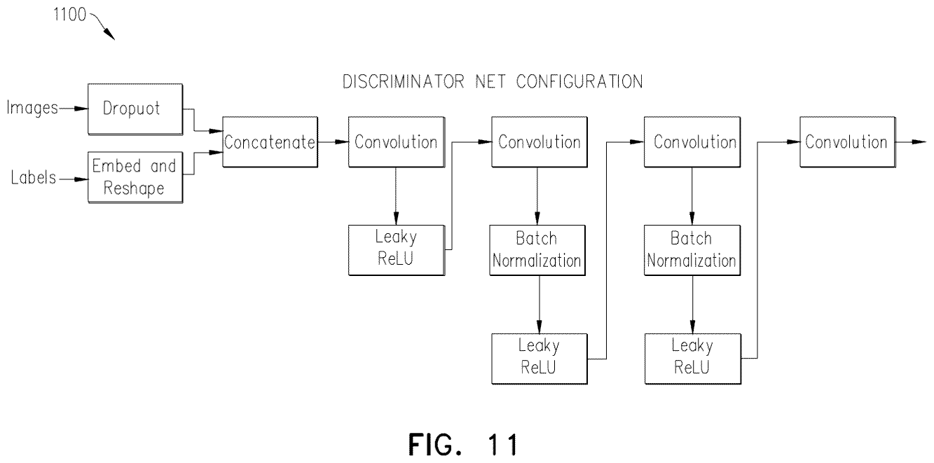

FIG. 11 is a schematic illustration of a discriminator net configuration, according to an embodiment of the disclosure;

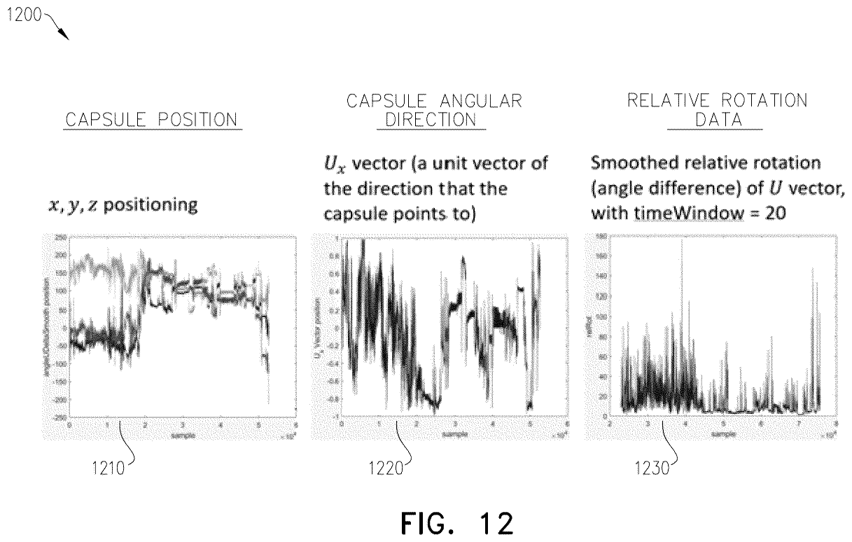

FIG. 12 is a schematic illustration of capsule position data, Capsule angular direction and relative rotation vector of an exemplary clinical case, according to an embodiment of the disclosure;

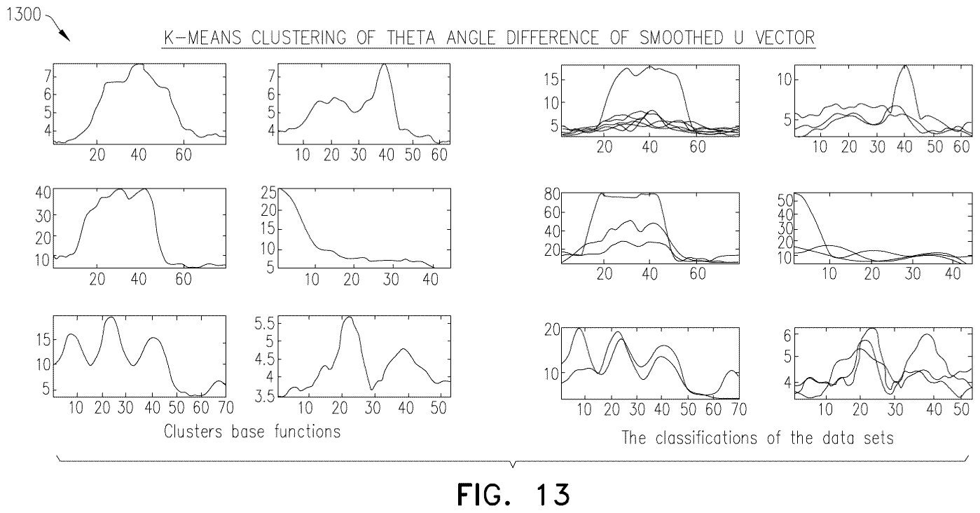

FIG. 13 is a schematic illustration of K-Means base vector decompositions of the relative rotation vectors for an exemplary clinical case. according to an embodiment of the disclosure; and

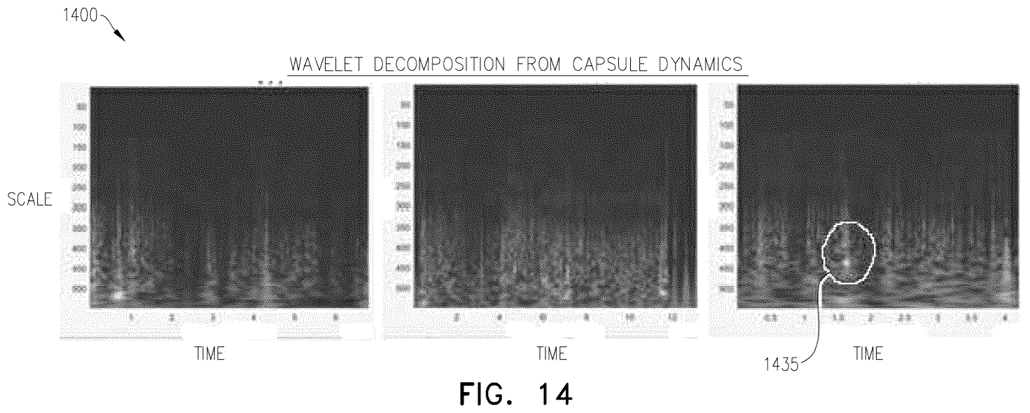

FIG. 14 is a schematic illustration of a wavelet decomposition from capsule dynamics, according to an embodiment of the disclosure.

### DETAILED DESCRIPTION

FIG. 1 is a schematic illustration of a perspective view of an imaging capsule 100 in a patient's colon. 190, according to an embodiment of the disclosure. Optionally, the imaging capsule 100 comprises a protective encasement 180 and is configured to flow through the gastrointestinal tract of a patient. In an embodiment of the disclosure, imaging capsule 100 includes a radiation source 110 that emits X-Ray or gamma radiation and is positioned at the center of a collimator 115 (e.g. a circular/cylindrical collimator) to control the direction of emission of radiation from the radiation source 110. Optionally, the radiation source is also located between two radiation blocking disks 125 (e.g. cylindrical tungsten disks) to prevent emission of radiation from the upper and lower ends of the imaging capsule 100. In an embodiment of the disclosure, as imaging capsule 100 traverses the colon it radiates the inner walls of the colon 190 with X-Rays and/or gamma radiation. In response one or more detectors 120 of imaging capsule 100 detect particles (e.g. photons, electrons) responding to the emitted radiation. Optionally, imaging capsule 100 forms a count for each energy level representing the number of particles having the specific energy level resulting from Compton backscattering and X-Ray fluorescence.

In an embodiment of the disclosure, the counts are provided as measurements of the detectors 120 to a computer 195 for processing. Optionally, the computer 195 may construct images of slices 140 of the colon 190 and/or construct images of an entire segment 150 (e.g. a continuum of slices 140) of the colon 190 or of the entire colon 190. In an embodiment of the disclosure, computer 195 includes an application 170 that uses a trained machine learning module 220 (FIG. 2), which accepts measurements and provides a determination for each slice 140 or segment 150 of the colon 190, whether the slice 140 or segment 150 comprises a polyp 160 or other abnormalities.

FIG. 2 is a schematic flow diagram of a process 200 of training 210 a machine learning module 220, according to an embodiment of the disclosure. In an embodiment of the disclosure, a data set comprising multiple instances of X-ray fluorescence measurements 202, Compton backscattering measurements 204, other data 206 (listed below) and a determination 208 if the respective slice 140 or segment 150 to which the data relates includes a polyp 160 or other abnormalities. Optionally, determination 208 includes two categories: category one—polyp (1), category two—non-polyp (0). Alternatively or additionally the determination 208 may be provided as a score (e.g. a probability value, 0% no polyp, 50% maybe, 100% Polyp or intermediate values). Optionally, a neural network is trained with the data set to form a machine learning (ML) module 220.

In an embodiment of the disclosure, ML, module 220 is configured to receive X-ray fluorescence measurements 202, Compton backscattering measurements 204 and optionally other data 206 for a slice 140 or segment 150 of slices 140. The ML module 220 analyzes the data and provide a score 230 indicating if a polyp 160 or other abnormalities are present in the slice 140 or segment 150. Optionally, the score may be a yes/no answer or may be a probability value as explained above.

In an embodiment of the disclosure, the other data 206 may include:

1. Pressure data measured within imaging capsule 100 representing the internal pressure of the gases and air within the capsule.
2. Capsule dynamics data, for example the average speed of the imaging capsule within the colon 190 or within the specific segment 150. Additionally, data such as capsule angular directions as a function of time, capsule 3D locations as a function of time, capsule position velocities and angular velocities and/or whole gut transfer time (WGTT)—the time it took for the capsule to pass through the patient's gastrointestinal tract. This information can provide an indication regarding the existence of a polyp causing the values to vary (e.g. slow down the imaging capsule 100 or cause it to turn).
3. Colonoscopy data that gives general information of the patient, for example if the patient was positive or negative for a polyp or a number of polyps in a colonoscopy examination, type of polyps found, estimated location of the polyps within the colon 190, size of the polyps. In addition, if the patient was positive or negative for colon cancer and if so, the location of the cancer.
4. Patient demographic information including age, sex/gender, BMI height, weight, medical history (e.g. previous polyps detected), smoking/non-smoking, first degree relatives with colon cancer, number of bowel movements per week, diet with vegetables, physical activity.

In an embodiment of the disclosure, the imaging capsule 100 scans consecutive slices of the colon inner surface, as it travels through the colon 190. The slice data are represented by photon counts of photons detected due to x-ray fluorescence 202 from a contrast material mixed with the colon contents and from Compton backscattering photons 204 that arrive from the colon contents, the colon tissue and from outside the colon. Following the analysis of this data, slices 140 representing the distance of the colon wall from the capsule are reconstructed. These slices 140 are stacked sequentially based on the time that they were acquired to form a pseudo 3D reconstruction of a segment 150. where the slice dimensions are in millimeters (min) and the Z direction along the route of the capsule is in the time dimension (seconds). Alternatively or additionally, raw signals from the photon detectors may be used as the input data for the classification algorithm used by the training module 210 and/or the ML module 220. Optionally, the data may be preprocessed. The preprocessing may include signal filtering such as low frequency filtering (LPF) detrending, fast Fourier transform (FFT). wavelet decomposing, Singular Value Decomposition (SVD), Principle Component Analysis (PCA) and other signal preprocessing prior to feeding the output of the measurements from the imaging capsule 100 to training module 210 and/or ML module 220 to classify the results.

In an embodiment of the disclosure, algorithms for the machine learning module 220 will in most cases not be trained with a determinate positive indication of polyp (1) or no-polyp (1) per specific 2D imaging slice. Only rarely will there be such accurate correlation since colonoscopy can rarely indicate such correlation with a good degree of certainty. It is therefore expected that a specific 2D slice 140 will generally not be labeled, rather only a segment 150 (a sequence of slices 140) will be provided with a determination 208.

Additionally, the other data that is not necessarily localized (per slice 140) such as pressure, motility dynamics, patient demographics etc. will be correlated to polyp (1) and non-polyp (0) for the entire colon 190 of the patient or for a segment 150, Likewise the existence of cancer or indications from a colonoscopy examination may be restricted to a certain colon sector/segment where the capsule dynamics, pressure or a combination of them in addition or as a complement to 2D slice data may point to a specific area—within the colon 190 where a polyp 160, a number of polyps 160 or cancer is present.

In an aspect of this disclosure, the ML module algorithm for classifying 2D scans as indicating a polyp (1) or non-polyp (0) will be generated from a set of algorithms that only use partial data which is labeled, and other data which is not labeled. For example some slices 140 may provide a clear indication and some may only, provide an intermediate probability value (e.g. 30% or 70%).

The types of classification algorithms that may be used for the 2D scans may be one of the following list: Linear classifier, Logistic Regression, Bayes linear, Bayes quadratic, nearest neighbor, kn-Nearest neighbor, Parzen window, Neural network, Decision tree, Support Vector Machines, Boosted Trees, Random Forest, and Stochastic Gradient Descent. Additionally, algorithms such as artificial neural network (ANN) such as a Generative Adversarial Network (GAN) may be used as a semi-supervised ANN.

In an embodiment of the disclosure, training is based on human knowledge in addition to confirming with colonoscopy when applicable. A large number of 2D scans from patients with known polyps and patients with a known clean colon 190 are fed as input vectors (a training data set) to the training module 210. Optionally, an interactive system requests a human expert to give scores between 0 and 100 correlated to the probability of a 2D scan slice 140 comprising a polyp 160. The expert inputs are fed to a list of expected outcomes to form a labeled data set. Following such an interactive process, a clustering algorithm begins to work with the aim to find a best set of features in the data that will separate the data into polyp and non-polyp by setting thresholds to the score and trying to maximize the area under the curve (AUC) of a Receiver Operator Curve (ROC) generated by this process.

FIG. 3 is a schematic illustration of a lab generated set 300 of polyp and non-polyp 2D maps produced from capsule imaging data; according to an embodiment of the disclosure. The lab generated set 300 includes a map based on X-ray fluorescence, a map based on Compton backscattering and a map based on the two fused together.

FIG. 4 is a schematic illustration 400 of an exemplary set of a Receiver Operator Curve (ROC) and performance of a human expert classifier. The ROC shows a clear separation between slices with suspect polyps and slices with no polyps. The ROC shows good Sensitivity and specificity performance of the classifying algorithm which enables the detection of polyps and non-polyps based on a single imaging slice. The lower part of FIG. 4 shows classification criteria depicting roundness relative to a protrusion score. Optionally, the less round the map and the higher the protrusion score indicates that there is more likelihood of having a polyp.

Following the completion of the first set of human expert guided classifications, the classifier having optimized the feature vector set for optimal. AUC, runs an additional large set of new data that includes both polyp and non-polyp 2D segments and classifies this data with the feature vector that was optimized in the process with the human expert. At this stage, the amount of data is much larger. To validate the process, the algorithm presents the human expert with randomly selected 2D slices and asks the human expert to give a score between 0 and 100. The algorithm compares this new randomly selected human expert test score vector with a test score vector generated by the classification algorithm. Based on the difference between the two test score vectors, a new feature set is chosen by the algorithm taking the average of the 2 sets. Another run is made on the data of a new set of randomly chosen polyp and non-polyp 2D scans (based on the classifier scores). The results are presented to the human expert and a new set of human expert scores are recorded and compared with the classifier scores. In order to optimize this process of training by a human expert and understanding that the human expert may have difficulty giving the intermediate scores, or he may opt in a lot of cases to give a score of about 50—undecided. These scores will be taken with low weight and the scores of near 0 or near 100 will be taken with a high weight factor. Alternatively, or additionally, during the human expert interactive process, the system will present some of the 2D slices repeatedly and average the score it got for that slice from the human expert.

In an aspect of this disclosure Consecutive 2D slices will be presented to the human expert and his decision on the score for a particular 2D slice will be based on viewing the slices preceding and following that slice. The same process as described above will be performed but the human expert will use 2D slices before and after the chosen slice in order to evaluate the probability that the slice image is a polyp. For the classification algorithm evaluation, it will include also the slices preceding and following the selected slice and the process will be similar to the one described above, only that a few slices will be evaluated together and the feature vector will also include parameters related to the correlation between slices and not only for the slice data itself.

In an embodiment of the disclosure, 2D and 3D maps of sectors of the colon are presented to an expert human and the process far evaluating the probability for the presence of a polyp in the chosen sector is performed in a similar manner to the process described above for a single 2D slice and a small set of 2D slices. FIG. 5 is a schematic illustration 500 of 2D 520 maps and 3D images 530 of polyps, the image of the polyp in a colonoscopy procedure 540 and the 2D sector slice of the polyp 510, according to an embodiment of the disclosure.

In an embodiment of the disclosure, a semi-supervised clustering algorithm is used to optimize the decision between a polyp and non-polyp based on a small amount of labeled data based on human expert decisions and colonoscopy findings that a polyp is detected vs a large amount of unlabeled data where the algorithm categorizes . the data into polyp and non-polyp findings.

For this algorithm, the data can be 2D slices 510, 2D Map's 520 or 3D images 530 as described above for the human expert algorithm. Additionally, capsule dynamics data such as capsule angular directions as a function of time, capsule 3D locations as a function of time, capsule position velocities and angular velocities are also considered to be part of the data for polyp detection as an additional set of data vectors for training the ML algorithm or as a separate set of data vectors for a semi supervised ML algorithm.

To understand the semi supervised ML algorithm, let us assume a set of data inputs, {x1 . . . xn} and a small set of labeled data outputs corresponding to the data inputs {y1 . . . }— where m<<n. The following are assumed for the input data and corresponding outputs, labeled or not labeled..

Continuity Assumption

Points that are close to each other are more likely to share a label, This is also generally assumed in supervised learning and yields a. preference for geometrically simple decision boundaries. In the case of semi-supervised learning, the smoothness assumption additionally yields a preference for decision boundaries in low-density regions, so few points are close to each other but in different classes.

Cluster Assumption

The data tend to form discrete clusters, and points in the same cluster are more likely to share a label (although data that shares a label may spread across multiple clusters). This is a special case of the smoothness assumption and gives rise to feature learning with clustering algorithms.

### Manifold Assumption

The data lie approximately on a manifold of much lower dimension than the input space. In this case learning the manifold using both the labeled and unlabeled data can avoid the curse of dimensionality. Then learning can proceed using distances and densities defined on the manifold.

The manifold assumption is practical when high-dimensional data are generated by some process that may be hard to model directly, but which have only a few degrees of freedom. For instance, human voice is controlled by a few vocal. folds, and images of various facial expressions are controlled by a few muscles. In These cases, distances and smoothness in the natural space of the generating problem, is superior to considering the space of all possible acoustic, waves or images, respectively.

Several possible ML algorithm models may be applied for the semi-supervised ML algorithm to categories between polyp (1) and non-polyp (0) data.

FIG. 6B depicts a table 600 depicting the concept of mixing supervised and unsupervised ML algorithms with their respective data sets to create a combined algorithm..

1. A semi-supervised machine-learning, algorithm uses a limited set of labelled sample data to train itself, resulting in a ‘partially trained’ model.

2. The partially trained model labels the unlabeled data. Because the sample labelled data set has many severe limitations (for example, selection bias in real-world data) the results of labelling are considered to be ‘pseudo-labelled’ data.

3. Labelled and pseudo-labelled datasets are combined, creating a unique algorithm that combines both the descriptive and predictive aspects of supervised and unsupervised learning.

Semi-supervised learning uses the classification process to identify data sets and clustering process to group it into distinct parts.

FIG. 6A is a flow diagram of a method 610 of training a machine learning module 220, according to an embodiment of the disclosure. In an embodiment of the disclosure, computer 195 receives (615) a first data set of X-ray fluorescence measurements 202, Compton backscattering measurements 204, and optionally other data 206 related to a slice 140 or segment 150 of slices 140 of colon 190. Optionally, a human expert or dedicated expert program is used to make an initial attempt to classify (620) the data and provide a polyp determination 208 as part of the data set.

In an embodiment of the disclosure, the data set is used by a neural network to train (625) a first machine learning module 220 to identify if the X-ray fluorescence measurements 202, Compton backscattering measurements 204, and other data 206 indicate the existence of a polyp 160 or not. The first version of the machine learning module 220 is then used to classify (630) a new data set. Optionally, the new data set is much larger than the initial data set, since the first set may be prepared by a human expert or an expert program with limited ability (e.g. capable of identifying cases that meet specific rules) while the new data set is already prepared by a machine learning module that is capable to automatically improve itself.

In an embodiment of the disclosure, the expert checks (635) classifications of at least sonic of the cases from the new data set to determine if the trained machine learning module 220 is capable of providing good quality classifications. Optionally, the checked cases may be selected randomly or for example selected in order as a percentage of the cases (e.g. 0.1%, 0/5% or 1%). In an embodiment of the disclosure, the checked classification are compared (640) with the classification provided by the machine learning module 220 and any discrepancies are taken into consideration while training (645) an improved machine learning module 220. Optionally, if the number of discrepancies is below a preselected threshold value then the machine learning module 220 is assumed to be have a good classification level. Otherwise if the classification quality needs improvement (650) then we can repeat classifying (630) a new data set with the current trained machine learning module 220 until we achieve a machine learning module that provides satisfactory results, which can be used (655) reliably.

Algorithm: Semi-Supervised Generative Adversarial Network (GAN)

The Semi-Supervised GAN, abbreviated as SGAN for short, is a variation of the Generative Adversarial Network architecture to address semi-supervised learning problems. The following is a description of this algorithm that was developed to deal with the specific properties of the X-ray imaging capsule 100 including several unique features specific only for an X-ray imaging capsule 100.

In an embodiment of this disclosure, a Semi-Supervised GAN classifier for an X-ray imaging capsule is implemented as follows

A large set of unlabeled data is fed into a deep artificial neural network (ANN) and in parallel, an ANN generator that has at its input, white noise vectors with normal distribution, uniform distribution or other distribution which is representative of the data from the X-ray imaging capsule data (this data can be imaging data as well as capsule dynamics data as described above). See FIG. 7 illustrating such a process.

Both neural networks are trained in parallel with a classifier as the outcome parameter that needs to decide if the input is a true image (capsule dynamics data representing a polyp) from the capsule or a fake image from the generator (fake capsule dynamics from the generator). Equation 1 describes such a Min/Max optimization process.

Min _{G} Max _{D} L(D,G)=E_{x˜Pr(x)}[logD(x)]+E_{2˜Pz(z)}[log(1−D(G(z)))] Equation 1

The optimization described in Equation 1 consists of the sum of 2 contradicting processes. The first part of the equation (see also FIG. 8—the discriminator artificial neural network (ANN)) is the discriminator Irving to best recognize a true image and reject fake generated images from the ANN fake image generator. The second part of the equation tries to best generate a fake image to fool the discriminator. By letting these two “adversaries” to train against each other, the discriminator ANN and the generator ANN will “learn” the important features of the images (capsule dynamics features representing a polyp)

The algorithm optimizes D, to maximize the probability to assign the conect label (true or fake data) and simultaneously optimizes G, to minimize the probability to assign the correct label (true or fake data).

After the algoritlun is trained with unlabeled data, it will converge to recognize fake vs. real data. Two (2) additional categories of Polyp (1) and non-polyp (0) are also part of this ANN output and at this stage of the training with the unlabeled data, are not given expected outputs.

Following the training of the ANN discriminator and the ANN generator to distinguish between true and fake data, the ANNs are expected to have a partial representation of the main data features. At this stage, labeled data is introduced along with an output per label of polyp (1) or non-polyp (0). Now the discriminator ANN is rim again with a large set of unlabeled data and randomly distributed—within this unlabeled data, labeled data. When an unlabeled data is entered, only the weights for the fake/real part of the ANN are allowed to be changed with the algorithm from the ANN generator as a reference. When labeled data is entered, all weights can be changed. The last layer contains the weights for the 3 output categories.

Following this training, the last layer of the discriminator ANN is expected to learn how to separate the polyp (1) vs. non-polyp (0) categories as well as the fake vs. real images andfor capsule dynamics data.

In another embodiment of this disclosure a conditional GAN (cGAN) is used to improve the performance of the categorization of the GAN. This cGAN algorithm is designed with the special requirements of the x-ray imaging capsule and the labeled data from colonoscopy that has limited position localization correlation capability. For the cGAN algorithm to function, a generator net configuration 1000 is designed as depicted in FIG. 10 and a discriminator net configuration 1100 is designed as depicted in FIG. 11, FIG. 9 depicts the overall configuration of the cGAN algorithm net configuration 900 with both the generator net and the discriminator net.

The cGAN 900 is trained by inputting , data vectors at the input to the discriminator and random data with similar statistical distribution as the real data vectors at the generator input. Data fed to the discriminator may be x-ray fluorescence photon counts, Compton backscattering photon counts, capsule position data, capsule orientation data, capsule internal pressure data and any combination of these data. Alternatively or additionally, preprocessing may be done on the data such as LPF FFT Wavelet transforms, PCA, SVD and other analysis may be performed on the data before inputting to the cGAN algorithm.

For the cGAN 900 algorithm, additional 7 classes are added as conditional inputs both to the discriminator and generator. These are integers 1-7 and function as conditional inputs. These classes are 1-4 corresponding to a polyp found by colonsocopy its 1-4 sectors of the colon (1=Cecum-Ascending, 2=Transverse, 3=Descending Sigma, and 4=Rectum. 5=A polyp in the colon but with no known position, 6=Colonoscopy did not find any polyps in the colon, 7=Colonoscopy not performed, no result).

That is, for each of these classes, the class input is mapped to a unique vector both in the generator net and in the discriminator net. Thus, the generator tries to generate a “fake” data set for a particular conditional class and the discriminator tries to discriminate between fake and true for the chosen class. Additionally, since the capsule position changes over the course of its travel. through the colon, it will be positioned in one of the 4 colon sectors for any particular data set. The sector of the capsule findings will be regarded as relevant to the colonoscopy localized findings (class 1-4) if the capsule position and the colonoscopy position are correlated or 1 segment adjacent to each other, thus allowing for position inaccuracies. In the case that the colonoscopy findings arc without location, the classification will be “Patient with polyp”. In case that the colonoscopy result is negative, the class of negative result will be conditioned “Patient with no polyp”. In case that there is no colonoscopy data, that is an unsupervised data set with no known result. In these cases, which may be most of the data for the training of the cGAN algorithm, all the weights connecting the conditional vector are not changed for these data sets. Nevertheless, other weights in generator and discriminator are allowed to change and this allows the system to leans to train on the general features of the data, while in the cases that the colonoscopy results are known, the additional information allows improved training by directing the net to a specific category. It should be noted that there will be cases when more than one category is applicable simultaneously, for example, when a colonoscopy result is positive for one or more colon segments as there ma be polyps in more than one colon segment, then the relevant categories that the eGAN should show positive will be for example category 1—polyp in the Cecum ascending colon sector and category 5—a polyp in the colon. On the other hand, when category 6—no polyp in the colon is positive, then no other categories are expected to be positive.

In an embodiment of the disclosure, additional data parameters related to the patient may be added such as age, smoking/non-smoking, BMI, Whole Gut Transit Time (WGTT) of the capsule, Gender, polyps found in past colonoscopy, first degree relative with colon cancer yes/no, No. of bowel movements per week, diet with vegetables yes/no, physical activity Yes/No. This data in the form of an input data vector may be added per patient to the data inputted to the net. It is known in the literature that this data correlates with the probability of colon cancer and therefore the cGAN net may find that this data can improve the categorization of patients with and without polyps.

In an embodiment of the disclosure, recurrent ANN is trained to categorize capsule motility data and pressure data, or this data after post preprocessing. A global parameter for the training is the result of a colonoscopy on a per patient basis. The outputs of the recurrent ANN are two (2) categories polyp (I) and non-polyp (0). In most cases, this global labeling is available, but localization of the polyp may not be available. In order to train a recurrent ANN with this type of capsule dynamics data, the data is divided into 4 or 5 colon “segments”. A preprocessing phase may be used to shorten the training time and data size requirements by using heuristic knowledge such as inputting capsule velocities, capsule direction vectors, capsule transit times and other data that was found to be correlated to the presence of polyps in the colon. Additional preprocessing may be in the frequency domain performing FFT transforms on the data, performing wavelet decomposition and other frequency domain analysis. Other preprocessing may be SVD and PCA analysis of other mathematical analysis to reduce the dimensionality of the data as a step before training is applied.

In an embodiment of the disclosure, motility data such as 3D position data, vectorized capsule velocity data, angular directional capsule data and other such motility data is used in an algorithm that separates these data. sets to intervals that are then quantified into a set of base functions using algorithms such as K-Means, Wavelet decomposition and other such base functions. These base functions are then used to divide the motility data into clusters and separate the clusters based on colonoscopy data in the same patients into (0) no-polyp clusters and (I) polyp clusters based on features in the cluster sets.

In an embodiment of the disclosure, the base functions described above such as K-Means data vectors, wavelet decomposition base functions or other base functions that span the motility data vectorized data set are stacked in a 2D image like format. This 2D format that forms a 2D “linage” is then analyzed with machine learning (ML) that are used to cluster images to find features that indicate the presence of polyps or no-polyps in the data set.

FIG. 12 shows capsule position data (X,Y,Z) 1210 Capsule angular direction 1220 and relative rotation vector 1230 of an exemplary clinical case.

FIG. 13 shows a K-Means base vector decompositions 1300 of the relative rotation vectors for the exemplary clinical case.

FIG. 14 shows examples of wavelet decomposition from capsule dynamics 1400 showing “features” 1435 in the 2D representation of the capsule motility data. Such features 1435, or a combination of such features 1435 can be correlated to the presence of polyps in the colon.

In another embodiment of this disclosure, the machine learning algorithms described above that are not ANNs may use the same data sets as described for the ANNs.

In an embodiment of the disclosure, a recurrent neural network (RNN) is used to train the network to recognize slices which correlate to the presence of polyps vs slices with no polyp. This sequential correlation, for which recurrent ANNs are specially designed to identify, can enhance the performance of a classifier of which, the RNN is a part of.

In an embodiment of the disclosure, both X-Ray Fluorescence (XRF) data. 202 and Compton Backscattering (CMT) data. 204 are used for the classification of the data as polyp (1) non-polyp (0) are used. The use of these two types of signals instead of a single signal, enables discrimination between real polyps and air bubbles or other artifacts. Additionally, real polyps appear differently than artifacts and are well correlated between the XRF and the CMT data.

In an embodiment of the disclosure, the distribution of the X-ray contrast material along the colon length may be used as indicators of colon malignancies, by factoring the distribution in conjunction with other parameters such as capsule motility and pressure measurements along the colon route, the presence of polyps, colon cancer as well as indications of other illnesses such as IBS, IBD, Crohn's disease and other inflammations in the gastrointestinal track may be diagnosed.

In an embodiment of the disclosure, the calculated diameters of the colon and their distribution along the colon length can give a correlative indication as to the presence of polyps, colon cancer as well as indications of other illnesses such as IBS, IBD, Crohn's disease and other inflammations in the gastrointestinal track may be diagnosed.

In an embodiment of the disclosure, the system accumulates statistics of different features and groups of features, so that when a suspect is examined, the statistics of features for the suspect are presented to the human expert. This can aid in separating between common features such as anatomical features in the colon and rare features which may be polyps or other lesions that are not seen often in the colon.

In an embodiment of the disclosure, the system allows for an iterative process between the human expert and the machine learning algorithm to continuously learn from the human expert on suspected polyps and improve the classification algorithm.

It should be appreciated that the above described methods and apparatus may be varied in many ways, including omitting or adding steps, changing the order of steps and the type of devices used. It should be appreciated that different features may be combined in different ways. In particular, not all the features shown above in a particular embodiment are necessary in every embodiment of the disclosure. Further combinations of the above features are also considered to be within the scope of some embodiments of the disclosure.

It will be appreciated by persons skilled in the art that the present invention is not limited to what has been particularly shown and described hereinabove. Rather the scope of the present invention is defined only by the claims, which follow.

I/We claim:

1. A system for gastrointestinal examination, comprising:
an imaging capsule configured to scan the gastrointestinal tract of a patient using radiation and configured to measure X-ray fluorescence and Compton backscattering and transmit the measurements;
a computer with a processor and memory configured to receive the measurements from the imaging capsule;
a trained machine learning module executed by the computer configured to analyze the measurements and provide a probability score representing the probability of the existence of a polyp or other abnormalities in the gastrointestinal tract of the patient.

2. The system of claim 1, wherein the machine learning module considers also measurements of the internal pressure within the imaging capsule to calculate the probability score.

3. The system of claim 1, wherein the machine learning module considers also capsule dynamics to calculate the probability score; wherein the capsule dynamics are selected from the group consisting of: average speed within the colon, average speed within a specific segment, capsule angular direction as a function of time, capsule location as a function of time, capsule position velocity and capsule angular velocity.

4. The system of claim 1. wherein the machine learning module considers also colonoscopy information of the patient to calculate the probability score: wherein the colonoscopy information is selected from the group consisting, of if the patient had polyps in the past, if the patient has colon cancer, the whole gut transfer time of the imaging capsule.

5. The system of claim 1, wherein the machine learning module considers also demographic information of the patient to calculate the probability score; wherein the demographic information is selected hum the group consisting of: patient age, patient gender, BMI, height, weight, medical history, if the patient smokes, if the patient has first degree relatives that had cancer, number of bowel movements per week, if the patient eats vegetables.

6. The system of claim 1, wherein the machine learning module is trained by a neural network that is provided with a data set comprising multiple instances of X-ray fluorescence measurements and Compton backscattering measurements for a slice of the patients colon, with a determination if the measurements indicate that the slice comprises a poly.

7. The system of claim 1, wherein the machine learning module is trained by a neural network that is provided with a data set comprising multiple instances of X-ray fluorescence measurements and Compton backscattering measurements for a sequence of slices forming a segment of the patients colon, with a determination if the measurements indicate that the segment comprises a polyp.

8. The system of claim 7, wherein the neural network considers correlation between the slices in the sequence.

9. The system of claim 1, wherein the machine learning module is configured to discriminate between polyps and air bubbles based on the X-ray fluorescence measurements and Compton backscattering measurements.

10. The system of claim 1, wherein the machine learning module is trained by classifying a first data set with an expert classifier, training a first machine learning module, classifying a new data set with the first machine learning module, checking at least some of the classified new data set with the expert classifier and training an unproved machine learning module.

11. A method of examining a gastrointestinal tract, comprising:
scanning the gastrointestinal tract of a patient with an imaging capsule using radiation by measuring X-ray fluorescence and Compton backscattering and transmitting the measurements;
receiving the measurements from the imaging capsule by a computer with a processor and memory;
analyzing the measurements with a trained machine learning module executed by the computer and providing a probability score representing the probability of the existence of a polyp or other abnormalities in the gastrointestinal tract of the patient.

12. The method of claim 11, wherein thee machine learning module considers also measurements of the internal pressure within the imaging capsule to calculate the probability score.

13. The method of claim 11, wherein the machine learning module considers also capsule dynamics to calculate the probability score; wherein the capsule dynamics are selected from the group consisting of: average speed within the colon, average speed within a specific segment, capsule angular direction as a function of time, capsule location as a function of time, capsule position velocity and capsule angular velocity.

14. The method of claim 11, wherein the machine learning module considers also colonoscopy information of the patient to calculate the probability score; wherein the colonoscopy information is selected from the group consisting of: if the patient had polyps in the past, if the patient has colon cancer, the whole gut transfer time of the imaging capsule.

15. The method, of claim 11, wherein the machine learning module considers also demographic information of the patient to calculate the probability score; wherein the demographic information is selected from the group consisting of: patient age, patient gender, BMI, height, weight, medical history, if the patient smokes, if the patient has first degree relatives that had cancer, number of bowel movements per week, if the patient eats vegetables.

16. The method of claim 11, wherein the machine learning module is trained by a neural network that is provided with a data set comprising multiple instances of X-ray fluorescence measurements and Compton backscattering measurements for a slice of the patients colon, with a determination if the measurements indicate that the slice comprises a polyp.

17. The method of claim 11, wherein the machine learning module is trained by a neural network that is provided with a data set comprising multiple instances of X-ray fluorescence measurements and Compton backscattering measurements for a sequence of slices forming a segment of the patients colon, with a determination if the measurements indicate that the segment comprises a polyp.

18. The method of claim 17, wherein the neural network considers correlation between the slices in the sequence.

19. The method of claim 11, wherein the machine learning module is configured to discriminate between polyps and air bubbles based on the X-ray fluorescence measurements and Compton backscattering measurements.

20. The method of claim 11. wherein the machine learning module is trained by classifying a first data set with an expert classifier, training a first machine learning module, classifying a new data set with the first machine learning module, checking at least some of the classified new data set with the expert classifier and generating an in as learning module.
